(19) United States
(12) Patent Application Publication
(10) Pub. No.: US 2013/0217635 A1
(43) Pub. Date: Aug. 22, 2013

(54) NOVEL USE OF ERYTHROID DIFFERENTIATION REGULATOR 1 AS AN AGENT FOR TREATING CANCER

(71) Applicants: Dae Ho Cho, Seoul (KR); Sa Ik Bang, Seoul (KR); Jeong Min Park, Suwon-si (KR); Min Kyung Jung, Seoul (KR); Ha Rum Lee, Seoul (KR); Yoo Rim Park, Seoul (KR); Seung Beom Park, Seoul (KR); Juah Son, Seoul (KR); Youn Kyung Houh, Seoul (KR)

(72) Inventors: Dae Ho Cho, Seoul (KR); Sa Ik Bang, Seoul (KR); Jeong Min Park, Suwon-si (KR); Min Kyung Jung, Seoul (KR); Ha Rum Lee, Seoul (KR); Yoo Rim Park, Seoul (KR); Seung Beom Park, Seoul (KR); Juah Son, Seoul (KR); Youn Kyung Houh, Seoul (KR)

(73) Assignees: IL-YANG PHARM. CO., LTD, Gyeonggi-do (KR); Industry-Academic Cooperation Foundation, Sookmyun, Yongsan-gu, SEOUL (KR); SAMSUNGN LIFE WELFARE FOUNDATION, Seoul (KR)

(21) Appl. No.: 13/637,155

(22) PCT Filed: Mar. 23, 2011
(86) PCT No.: PCT/KR2011/001999

§ 371 Date: May 7, 2013

(30) Foreign Application Priority Data
Mar. 25, 2010 (KR) 10-2010-0026809

(51) Int. Cl. A61K 38/17

(52) U.S. Cl. CPC A61K 38/1709
USPC 514/19.3; 514/44 R; 435/7.92; 435/375; 435/455; 530/387.9; 436/501; 435/29

(57) ABSTRACT

The present invention relates to a novel use of erythroid differentiation regulator 1 (Erdr1) as an agent for treating cancer. More particularly, it relates to an use of Erdr1 or an expression vector including a polynucleotide encoding the same for preventing and inhibiting cancer metastasis, an use of Erdr1 or an expression vector including a polynucleotide encoding the same for preventing and treating cancer, an use of an antibody specific for Erdr1 for diagnosing cancer, or a method for screening agents for regulating cancer metastasis or cancer cells migration. The Erdr1 is negatively regulated by IL-18 expression and it suppresses migration, invasion and metastasis of cancer or tumor cell by expression of HSP90 and generation of ROI. And an Erdr1 recombinant protein promotes NK-cell killing activity against cancer cell. Accordingly, the Erdr1 and an expression vector comprising polynucleotide encoding the same and recombinant protein suppress cancer metastasis and bring an effect on activation of immune cells, and therefore can be useful for preventing, treating and diagnosing cancer.

### TECHNICAL FIELD

This application claims priority to Korean Patent Application No. 10-2010-0026809 filed on Mar. 25, 2010, which is hereby incorporated by reference herein.

The present invention relates to a novel use of erythroid differentiation regulator 1 (Erdr1) as an agent for treating cancer. More particularly, it relates to an use of Erdr1 or an expression vector including a polynucleotide encoding the same for preventing and inhibiting cancer metastasis, an use of Erdr1 or an expression vector including a polynucleotide encoding the same for preventing and treating cancer, an use of an antibody specific for Erdr1 for diagnosing cancer, or a method for screening agents for regulating cancer metastasis or cancer cells migration.

### BACKGROUND ART

Cancer is a complex disease which is caused by controlled growth and proliferation of transformed cells. Most of cancers occur due to mutation of oncogenes and tumor suppressor genes resulting from various causes including environmental and genetic factors. Cancer cells proliferate in the early stage, and then invade and destroy adjacent tissues. Gradually, they spread to the circulatory system and metastasize to distant locations in the body, and kill the subject in the end.

Many management options for treating cancer exist including surgery, radiation therapy, chemotherapy and other methods. Although many substances having anticancer effects isolated from various materials are used, most of the chemical anticancer agents exhibit toxicity to normal cells. Thus, development of new cancer treatment methods is constantly needed.

Furthermore, due to the metastasizing character of the cancer, i.e. the spread from the original location to other non-adjacent parts, many cancer patients fail to survive despite the advancement in surgery, radiation therapy, chemotherapy, or the like. Therefore, a new method capable of suppressing the metastasis of tumor cell is also required.

Melanoma is one of the most malignant skin tumors that have high mortality and metastatic characteristics. Despite the improved understanding of melanoma pathophysiology, the current immunological therapeutic approaches are still insufficient that increasement of death rate by melanoma. Metastasis involves multistep proceed by which cancer cells spread to distant sites to promote secondary colonies and induce cancer mortality. Cell motility including migration and invasion plays an important role in the process of metastasis. In melanoma, various factors are affected in the migration and invasion such as growth factors, chemokines and cytokines.

Interleukin-18 (IL-18) is an 18-kDa cytokine that belongs to the IL-1 cytokine superfamily. It is known to be an IFN-inducing factor and known as pro-inflammatory cytokines. IL-18 shows dual effects on cancer metastasis as anticancer factor and procancer factor. It produced various immune or non immune cells. It is reported that murine melanoma cell lines secrete IL-18, and endogenous IL-18 is associated with immune escape of murine melanoma cells by autocrine manners. IL-18 is one of the cytokine that related to induce the melanoma motility. Our previous study demonstrated that IL-18 enhanced migration ability occurs on B16F10 murine melanoma cells. This implies that positive correlation between enhanced IL-18 and malignant skin cancers, including melanoma, and this suggests important roles of IL-18 in the malignancy of skin tumors.

In addition, IL-18 production level is elevated by various stress which is required for IL-18 maturation and secretion. Several studies suggested that stressors enhance melanoma progression and induce escaping of immune surveillance system. It is also reported that psychological stress related hormone and various physical stress such as ROS and UV irradiation markedly promote melanoma metastasis.

Recently, it was newly demonstrated that erythroid differentiation regulator 1 (Erdr1) is released from cells under stress conditions. Erdr1 is first detected on mouse leukemia cell lines, moreover it expressed in many different normal murine tissues. Erdr1 show haemoglobin synthesis-inducing property.

Although both IL-18 and Erdr1 are known as stress related factors, the relationship of between IL-18 and Erdr1 is not understood until now. Furthermore, no information is available about the effect of Erdr1 in the regulation of the cancer metastatic process and related mechanism.

### DISCLOSURE

### Technical Problem

Accordingly, while the inventors of the present invention have carried out researches on the functions of Erdr1, we confirmed that Erdr1 decreased cell motility in vitro, and suppressed in vivo metastatic potential of murine melanoma cells via regulation of ROI generation and HSP expression, thereby completing the present invention.

Accordingly, the object of the present invention is to provide a novel use of Erdr1.

### Technical Solution

To achieve the above object, the present invention provides a composition for preventing and inhibiting cancer metastasis comprising Erdr1 as an effective ingredient.

To achieve another object, the present invention provides a composition for preventing and inhibiting cancer metastasis comprising an expression vector including a promoter and a polynucleotide encoding an Erdr1 polypeptide operably linked to the promoter.

To achieve another object, the present invention provides a composition for diagnosis of cancer comprising an antibody specific for Erdr1 polypeptide as an effective ingredient.

To achieve another object, the present invention provides use of Erdr1 polypeptide for preparing agents for preventing and inhibiting cancer metastasis and use of Erdr1 polypeptide for preparing agents for preventing and treating cancer.

To achieve another object, the present invention provides a method for preventing and inhibiting cancer metastasis, and preventing and treating cancer administering an effective amount of Erdr1 polypeptide to a subject in need thereof.

To achieve another object, the present invention provides use of an antibody specific for Erdr1 polypeptide for preparing agents for diagnosis of cancer.

To achieve another object, the present invention provides a method for diagnosis of cancer administering an effective amount of an antibody specific for Erdr1 polypeptide to a subject in need thereof.

To achieve another object, the present invention provides a method for screening agents for regulating for cancer metastasis or cancer cell migration.

Hereafter, the present invention will be described in more detail.

Erythroid differentiation regulator 1 (Erdr1) is produced in many tissue and its production is enhanced at stressful condition. This study investigated whether Erdr1 regulated murine melanoma progression, along with the mechanism involved in the Erdr1-regulated metastasis. In vitro, the level of cell migration and invasion ability was markedly inhibited by Erdr1-overexpression in B16F10 cells. To determine the regulated factors involved in Erdr1 suppressed cell motility, we measured the ROI levels. It was found that the ROI levels were increased by Erdr1 transfection. Because of heat shock protein (HSP) is also well known as stress protein and it contribute to cancer metastasis and invasion, we examined the HSP expression level in order to indentify the factors involved in Erdr1-reduced motility. HSP level was significantly decreased in Erdr1 overexpressed cells. It means that Erdr1 might inhibit the motility via inhibited generation of ROI and regulation of HSP. Due to cell motility is a key step in cancer metastasis, we further explore the putative anti-metastatic potential of Erdr1 in vivo, by injection of B16F10 cells transfected with or without Erdr1 into syngenic mice, C57BL/6. The group of injection with Erdr1 overexpressed cells significantly suppressed metastatic ability of melanoma and showed prolonged survival rate. Taken together, these results demonstrate that Erdr1 shows powerful anti-tumor effect, which has ability to reduce the metastatic potential of murine malignant melanoma cells.

Accordingly, the present invention provides a novel use of Erdr1 polypeptide for promoting apoptosis of cancer cell and regulation of cancer metastasis. More specifically, the present invention provides a novel use of Erdr1 polypeptide for apoptosis of cancer cell by NK cell or promoting/inhibiting cancer metastasis.

The present inventions provides a composition for preventing and inhibiting cancer metastasis comprising Erdr1 polypeptide as an active ingredient.

Meanwhile, the present invention provides a composition for preventing and inhibiting cancer metastasis comprising an expression vector including a promoter and a polynucleotide encoding an Erdr1 polypeptide operably linked to the promoter.

In addition, the present invention provides a composition for preventing and treating cancer comprising Erdr1 polypeptide as an active ingredient.

In addition, the present invention provides a composition for preventing and treating cancer comprising an expression vector including a promoter and a polynucleotide encoding an Erdr1 polypeptide operably linked to the promoter.

In addition, the present invention provides a composition for diagnosis of cancer comprising an antibody specific to Erdr1 polypeptide as an active ingredient.

Erdr1 of the present invention is Erythroid differentiation regulator 1 and it mostly exists as a dimer in vivo and some exist as a monomer or a tetramer and has a secretion character. Erdr1 of the present invention may be a well known Erdr1 protein (for example, Genbank Accession No. NP_{—}579940, CAA07729, CAD62281, AAH58113, AAH80795, AAH18296; EDL01287), but, preferably it may have an amino acid sequence represented by SEQ ID NO: 1 (NP_{—}579940). In addition, a polynucleotide encoding Erdr1 polypeptide may be a polynucleotide encoding well known Erdr1 polypeptide, but, preferably, it may have a nucleotide sequence represented by SEQ. ID NO: 2 (AJ539223). The above-disclosed Erdr1 protein is originated from mouse but it is disclosed that the human Erdr1 is identical to that of mouse (D P, et al., Cytokine, 2004, vol. 27(2-3), pp. 47-57)

The “promoter” of the present invention means a DNA sequence regulating the expression of nucleic acid sequence operably linked to the promoter in a specific host cell, and the term “operably linked” means that one nucleic acid fragment is linked to other nucleic acid fragment so that the function or expression thereof is affected by the other nucleic acid fragment. Additionally, the promoter may include a operator sequence for controlling transcription, a sequence encoding a suitable mRNA ribosome-binding site, and sequences controlling the termination transcription and translation. Additionally, it may be constitutive promoter which constitutively induces the expression of a target gene, or inducible promoter which induces the expression of a target gene at a specific site and a specific time, and examples thereof include a SV40 promoter, CMV promoter, CAG promoter (Hitoshi Niwa et al., Gene, 108:193-199, 1991; Monahan et al., Gene Therapy, 7:24-30, 2000), CaMV 35S promoter (Odell et al., Nature 313:810-812, 1985), Rsyn7 promoter (U.S. patent application Ser. No. 08/991,601), rice actin promoter (McElroy et al., Plant Cell 2:163-171, 1990), Ubiquitin promoter (Christensen et al., Plant Mol. Biol. 12:619-632, 1989), ALS promoter (U.S. patent application Ser. No. 08/409,297). Also usable promoters are disclosed in U.S. Pat. Nos. 5,608,149; 5,608,144; 5,604,121; 5,569,597; 5,466,785; 5,399,680; 5,268,463; and 5,608,142, etc.)

Examples of the vector of the present invention include a plasmid vector, a cosmid vector, a bacteriophage vector and a viral vector, but are not limited thereto. The preferred expression vector includes regulatory elements for gene expression such as a promoter, operator, an initiation codon, a stop codon, a polyadenylation signal, and an enhancer, and a variety of vectors can be prepared according to the purpose.

The polynucleotide of the present invention can be introduced into a target cell or host cell by inserting it as an phenotype by any method known in the art, such as infection, transfection or transduction.

As a host cell, a prokaryotic host cell such as Escherichia coli, Bacillus subtilis, Streptomyces, Pseudomonas, Proteus mirabilis or Staphylococcus, a lower eukaryotic host cell such as fungus (for example, Aspergillus), yeast (for example, Pichia pastoris, Saccharomyces cerevisiae, Schizosaccharomyces, Neurospora crassa), a cell originated from higher eukaryotic cell comprising an insect cell, a plant cell, a mammalian cell and so on may be used, but not limited thereto and preferably it may be a human cell and more preferably a cancer cell or tumor cell of human.

The composition of the present invention may be a pharmaceutical composition and the pharmaceutical composition of the present invention may be administered orally or parenterally. For oral administration, it comprise sublingual administration. Parenteral administration comprise injection method such as subcutaneous, intramuscular and intravenous injection and infusion. Erdr1 of the present invention or the expression vector thereof may be prepared as various pharmaceutical formula by mixing with a pharmaceutically acceptable carrier. The term “pharmaceutically acceptable” means what is physiologically acceptable and, when administered to human beings, generally does not cause allergic reactions, such as gastrointestinal disorder and dizziness, or similar reactions thereto. As a pharmaceutically acceptable carrier, in case of the oral preparations, binder, lubricant, solutionizer, exipient, solubilizer, dispersing agent, stabilizer, suspending agent, colorant and flavor may be used, in case of the injection, buffer, preservative agent, painkilling agent, solubilizer, isotonic agent and stabilizer may be mixed, and in case of a local administration reagent, exipient, lubricant and preservative agent may be used. As such, the formula of a pharmaceutical composition comprising Erdr1 of the present invention or the expression vector thereof may be prepared by mixing with a pharmaceutically acceptable carrier as described above. For example, in case of the oral administration, it may be prepared as a form of tablet, troche, capsule, elixir, suspension, gel, syrup, wafer and so on, and in case of the injection, it may be prepared as a form of a single dose formula or a multi dose formula. Preferably, the pharmaceutical composition of the present invention may comprise 0.0001-99.999 weight % of any one selected from group consisting of Erdr1 polypeptide, a polynucleotide encoding the same and an antibody specific for Erdr1, and 99.999-0.0001 weight % of a pharmaceutically acceptable carrier.

As used herein, the term “effective amount” refers to the amount showing effect on delivering agent to a subject, or on preventing, inhibiting, treating or diagnosing cancer metastasis and cancer diseases and as used herein, the term “subject” means animals, preferably, it means mammals, particularly animals including human beings and it may be a cell, tissue and organ originated from an animal. The subject may be patients in need of treatment.

Total effective amount of Erdr1 of the present invention or the expression vector thereof can be administered to a subject as a single dose, or can be administered using a fractionated treatment protocol, in which the multiple doses are administered over a more prolonged period of time. The content of the active ingredient in the pharmaceutical composition of the present invention can be varied depending on the severity of disease. however, usually, the effective amount of the composition may be administered once a day or multiple times a day with a effective dose of 0.1 to 100 mg/kg per body weight and more preferably 1 to 10 mg per body weight. However, the Erdr1 or the expression vector may be suitably determined by considering various factors, such as age, body weight, health condition, sex, disease severity, diet and excretion of a subject in need of treatment, as well as administration time and administration route. In view of these factors, any person skilled in the art may determine an effective dose suitable for the above-described specific use of the inventive polypeptide. The composition of the present invention has no special limitations on its formulation, administration route and administration mode as long as it shows the effects of the present invention.

Gene introducing method by using plasmid expression vector is the method which introduce plasmid DNA directly into a mammalian cell, and FDA has approved to use for human (Nabel, E. G., et al., Science, 249:1285-1288, 1990). Unlike viral vector, a plasmid DNA has advantage in respect of even purification. The acceptable expression plasmids of the invention may comprise mammalian expression plasmids which are used in the art. For example, but not limited thereto, pRK5 (European Patent No. 307,247), pSV16B (PCT Publication No. WO91/08291) and pVL1392 (PharMingen). The plasmid expression vector which comprise the said nucleic acid could be introduced to a target cell by, but not limited thereto, transient transfection, microinjection, transduction, cell fusion, calcium phosphate precipitation, liposome-mediated transfection, DEAE Dextran-mediated transfection, polybrene-mediated transfection, electroporation, gene gun, and other methods which are well known in the art (Wu et al., J. Bio. Chem., 267:963-967, 1992; Wu and Wu, J. Bio. Chem., 263:14621-14624, 1988).

The viral vectors which contain the nucleic acid may comprise, but not limited thereto, retrovirus, adenovirus, herpes virus, avipox virus, and lenti virus and the like. All of the viral genes of the said retroviral vectors were deleted or modified, and consequently non-viral proteins of the said vectors were produced by the infected cells. The main advantages of the retroviral vectors for gene therapy are to transfer large amount of genes into cloned cells, to integrate genes specifically which are transferred to cellular DNA, and to prevent additional infection after gene transformation (Miller, A. D., Nature, 357:455-460, 1992). The retroviral vectors which are approved by the FDA is manufactured by using PA317 amphotrophic retroviral packaging cell (Miller, A. D. and Buttimore, C., Molec. Cell Biol., 6:2895-2902, 1986). For the non-retroviral vectors, there is the said adenovirus (Rosenfeld et al., Cell, 68:143-155, 1992; Jaffe et al., Nature Genetics, 1:372-378, 1992; Lemarchand et al., Proc. Natl. Acad. Sci. USA, 89:6482-6486, 1992). The main advantages of the adenovirus are to transfer large molecular DNA fragment (36 kb), and to transfect non-cloned cells with very high titer. In addition, herpersviruses could be used in gene therapy for human (Wolfe, J. H., et al., Nature Genetics, 1:379-384, 1992).

In addition, Erdr1 of the present invention or polynucleotide encoding thereof may be administered by other methods, for example, locally, parenterally, orally, intranasally, intravenously, intramuscularly or subcutaneously, or by other suitable routes. Particularly, the Erdr1 or the expression vector thereof may be injected directly into a target cancer or tumor cell at an effective amount for treating the tumor cell. Particularly for a cancer or tumor present in a body cavity such as in the eye, gastrointestinal tract, genitourinary tract, pulmonary and bronchial system and so on, the inventive pharmaceutical composition can be injected directly into the hollow organ affected by the cancer or tumor using a needle, a catheter or other delivery tubes. Any effective imaging device, such as X-ray, sonogram, or fiberoptic visualization system, may be used to locate the target tissue and guide the needle or catheter tube. In addition, the inventive pharmaceutical composition comprising the nucleic acid encoding the AIMP2 protein may be administered into the blood circulation system for treatment of a cancer or tumor which cannot be directly reached or anatomically isolated.

For other pharmaceutically acceptable carriers, reference may be made to the following literature (Remington's Pharmaceutical Sciences, 19th ed., Mack Publishing Company, Easton, Pa., 1995).

In addition, the composition of the present invention may be administered in combination with the well known method or the compound for preventing or treating cancer. The well known method or the compound for preventing or treating cancer for combination with the composition of the present invention may be any one that is used for treatment of a tumor. For example, paclitaxel, doxorubicin, vincristine, daunorubicin, vinblastine, daunorubicin D, docetaxel, etoposide, teniposide, bisantrene, homoharringtonine, Gleevec (STI-571), 5-fluorouracil, Adriamycin, methotrexate, busulfan, chlorambucil, cyclophosphamide, melphalan, nitrogen mustard, nitrosourea, etc. may be included. The amount of the peptide of the present invention included in the composition of the present invention may be different depending on the kind and amount of the anticancer drug that the peptide binds to.

The diseases which can be applied the composition of the present invention may be cancers. The cancers comprise, but not limited thereto, malignant melanoma, leukemia, colon cancer, lung cancer, liver cancer, stomach cancer, esophagus cancer, pancreatic cancer, gall bladder cancer, kidney cancer, bladder cancer, prostate cancer, testis cancer, cervical cancer, endometrial carcinoma, choriocarcinoma, ovarian cancer, breast cancer, thyroid cancer, brain tumor, head or neck cancer, skin cancer, lymphoma and aplastic anemia. The lymphoma comprise B-cell neoplasms such as Precursor B-cell neoplasm, T-cell and NK-cell neoplasms such as Precursor T-cell neoplasm and Hodgkin lymphoma (Hodgkin disease) such as Classical Hodgkin lymphoma.

In addition, the pharmaceutical composition of the present invention may comprise one or more buffers (for example, saline or PBS), carbohydrate (for example, glucose, mannose, sucrose, or dextran), stabilizer (for example, sodium hydrogen sulfite, sodium sulfite or ascorbic acid), antioxidant, bacteriostat, chelating agent (for example, EDTA or glutathione), adjuvant (for example, aluminium hydroxide), suspension agent, thicking agent and/or preservative (benzalkonium chloride, methyl- or propyl-paraben and chlorobutanol) additionally.

In addition, the pharmaceutical composition of the present invention may be formulated to produce quick, durable or delayed release of an active component after administered to mammals using the method well known in the art.

In addition, the present invention provides an use of Erdr1 polypeptide for preparing agents for preventing and inhibiting cancer metastasis. In addition, the present invention provides a method for preventing and inhibiting cancer comprising administering an effective amount of Erdr4 polypeptide to a subject in need thereof.

In addition, the present invention provides an use of Erdr1 (erythroid differentiation regulator 1) polypeptide for preparing agents for preventing and treating cancer. In addition, the present invention provides a method for preventing and treating cancer comprising administering an effective amount of Erdr1 polypeptide to a subject in need thereof.

And the composition of the present invention may be a composition for diagnosis of cancer comprising an antibody specific Erdr1 polypeptide. And the “antibody” refers a specific protein molecule that targets an antigenic region. The antibody used therein may be, but not limited thereto, a monoclonal, a polyclonal antibody, an immunological active fragment (for example, Fab or (Fab)2 fragment), an antibody heavy chain, a humanized antibody, an antibody light chain, genetically manipulated single chain Fv molecule and a chimeric antibody.

The antibody of the present invention may be prepared by the method well known in the immunological field. Erdr1 protein used as an antigen of the present invention may be well know Erdr1 protein (for example, Genbank Accession No. NP_{—}579940, CAA07729, CAD62281, AAH58113, AAH80795, AAH18296, EDL01287), but preferably it may have an amino acid sequence represented by SEQ ID NO: 1 (NP_{—}579940).

Polyclonal antibodies may be prepared by injecting the Erdr1 protein into an animal and collecting blood samples from the animal to obtain serum containing antibodies, and monoclonal antibodies may be prepared by a method widely known in the art, such as a hybridoma method (Kohler and Milstein, European Journal of Immunology, 6:511-519 (1976)) or a phage antibody library technique (Clackson et al, Nature, 352:624-628 (1991); and Marks et al, J. Mol. Biol., 222:58, 1-597 (1991)).

For a method for determining the expression of Erdr1 protein of the present invention, various immunological analysis methods which are well known in the art can be used. The immunological analysis methods may be comprised whatever the method can measure binding of antigen-antibody complex. The method has been well known in the art, and for example, there are immunocytochemistry and immunohistochemistry, radioimmunoassays, ELISA(Enzyme Linked Immunoabsorbent assay), immunoblotting, Farr assay, immunoprecipitation, latex aggregation, erythrocyte aggregation, nephelometry, immunodiffusion, counter-current electrophoresis, single radical immunodiffusion, protein chip and immunofluorescence.

The “antigen-antibody complex” means a binding complex of Erdr1 protein and an antibody specifically recognizing thereof.

In addition, the present invention provides an use of an antibody specific for Erdr1 polypeptide for preparing diagnostic agents for cancer. In addition, the present invention provides a method for diagnosis of cancer administering effective amount of an antibody specific for Erdr1 polypeptide to a subject in need thereof.

In addition, the present invention provides a method for screening agents for regulating cancer metastasis or cancer cell migration comprising:

(a) contacting Erdr1 to a test agent in the presence of an test reagent;

(b) selecting the test agent which change Erdr1 activity by measuring Erdr1 activity; and

(c) testing whether the selected agent regulates cancer metastasis or cancer cell migration.

Various biochemical and molecular biology techniques or assays well known in the art can be employed to practice the screening method of the present invention. Such techniques are described in, e.g., Sambrook et al., Molecular Cloning: A Laboratory Manual, Cold Spring Harbor Press, N.Y., Second (1989) and Third (2000) Editions; and Ausubel et al., Current Protocols in Molecular Biology, John Wiley & Sons, Inc., New York (1987-1999).

Preferably, the test agent is first assayed for their ability to modulate a biological activity of an Erdr1 (“the first assay step”). Particularly, in the first step, modulating agents that modulate a biological activity of an isolated Erdr1 polypeptide may be identified by assaying a biological activity of isolated Erdr1 in the presence of a test agent. More preferably, the present invention may comprise:

(a) contacting Erdr1 to a test agent in the presence of the test agent;

(b) selecting the test agent which change activity of Erdr1 by measuring activity of Erdr1.

Regulation of different biological activities of the Erdr1 polypeptide can be assayed in the first step. For example, a test agent can be assayed for activity to modulate expression level of the Erdr1 polypeptide, for example, transcription or translation. The test agent can also be assayed for activities in modulating cellular level or stability of the Erdr1 polypeptide, for example, post-translational modification or proteolysis.

Test agents that increase a biological activity of the Erdr1 polypeptide by the first assay step are identified, the test agents are then subject to further testing for ability to express of IL-18, further regulate cancer metastasis or cancer cell migration in the presence of the Erdr1 (“the second testing step”). For example, the test agents are then subject to further testing for ability to regulate cancer metastasis or cancer cell migration.

On the other hand, if a test agent modulates an activity other than cellular level of the Erdr1, then the further testing step is needed to confirm that their modulatory effect on the Erdr1 would indeed lead to regulate cancer metastasis or cancer cell migration. For example, a test agent, which modulates phosphorylation activity of an Erdr1, needs to be further tested in order to confirm that modulation of phosphorylation activity of the Erdr1 can result in regulation cancer metastasis or cancer cell migration.

In both the first assaying step and the second testing step, either an intact Erdr1 and their fragments, analogs, or functional derivatives can be used. The fragments that can be employed in these assays usually retain one or more of the biological activities of the Erdr1. And fusion proteins containing such fragments or analogs can also be used for the screening of test agents. Functional derivatives of Erdr1 have amino acid deletions and/or insertions and/or substitutions while maintaining one or more of the bioactivities and therefore can also be used in practicing the screening methods of the present invention.

A variety of well-known techniques can be used to identify test agents that modulate Erdr1. Preferably, the test agents are screened with a cell based assay system. For example, in a typical cell based assay for screening p53 modulators (i.e., the second screening step), a construct comprising a p53 transcription regulatory element operably linked to a reporter gene is introduced into a host cell system. The activity of polypeptide encoded by the reporter gene (i.e., reporter polypeptide), e.g., an enzymatic activity, in the presence of a test agent can be determined and compared to the activity of the reporter polypeptide in the absence of the test agent. An increase or decrease in the activity identifies a modulator of p53. The reporter gene can encode any detectable polypeptide (response or reporter polypeptide) known in the art, e.g., detectable by fluorescence or phosphorescence or by virtue of its possessing an enzymatic activity. The detectable response polypeptide can be, e.g., luciferase, alpha-glucuronidase, alpha-galactosidase, chloramphenicol acetyl transferase, green fluorescent protein, enhanced green fluorescent protein, and the human secreted alkaline phosphatase.

In the cell-based assays, the test agent (e.g., a peptide or a polypeptide) can also be expressed from a different vector that is also present in the host cell. In some methods, a library of test agents is encoded by a library of such vectors (e.g., a cDNA library). Such libraries can be generated using methods well known in the art (see, e.g., Sambrook et al. and Ausubel et al., supra) or obtained from a variety of commercial sources.

In addition to cell based assays described above, modulators of p53 can also be screened with non-cell based methods. These methods include, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel et al., supra (chapter 12, DNA-Protein Interactions). One technique for isolating co-associating proteins, including nucleic acid and DNA/RNA binding proteins, includes use of UV crosslinking or chemical cross-linkers, including e.g., cleavable cross-linkers dithiobis (succinimidylpropionate) and 3,3′-dithiobis (sulfosuccinimidyl-propionate); see, e.g., McLaughlin, Am. J. Hum. Genet., 59:561-569, 1996; Tang, Biochemistry, 35:8216-8225, 1996; Lingner, Proc. Natl. Acad. Sci. U.S.A., 93:10712, 1996; and Chodosh, Mol. Cell. Biol., 6:4723-4733, 1986.

In an example of the present disclosure, it was investigated whether the expression of Erdr1 is regulated by IL-18 in the mouse melanoma cells B16F10, using B16F10 cells transfected with IL-18 antisense (B16F10/IL-18 antisense transfectants). As a result, it was found out that the expression of Erdr1 is negatively regulated by IL-18, since the Erdr1 expression remarkably increased in the B16F10/IL-18 antisense transfectants as compared to the wild type B16F10.

In another example of the present disclosure, it was revealed that Erdr1 might be a potential suppressor of melanoma migration, since the mouse melanoma cells B16F10 in which Erdr1 is overexpressed showed reduced capacity to migrate and invade.

In another example of the present disclosure, experiment was carried out to find a factor involved in the regulation of cell migration by Erdr1. As a result, it was revealed that Erdr1 is closely related to ROI signal transduction, since the melanoma cells in which Erdr1 is overexpressed showed suppressed expression of heat shock protein 90 and reduced ROI level.

In another example of the present disclosure, metastasized tumors of C57/BL6 syngenic were counted in order to investigate whether Erdr1 is effective for melanoma metastasis in vivo. As a result, it was revealed that the overexpression of Erdr1 suppresses metastasis of cancer cells and survival time.

In another example of the present disclosure, the effect of the Erdr1 protein on natural killer cells and cytotoxicity related thereto, particularly cytotoxicity against cancer cells. As a result, it was revealed that the Erdr1 protein is capable of improving the ability to kill cancer cells by mediating degranulation of the natural killer cells.

In another example of the present disclosure, it was found out by an immunohistochemical method that the Erdr1 protein is expressed in normal tissue cells but not in the tissue cells of patients. Thus, it was verified that an antibody specific for the Erdr1 protein can be used for diagnosis of cancer.

Hereafter, the drawings of the present invention are described in detail.

FIG. 1 shows Erdr1 level increased on B16F10 antisense IL-18.

Compare of Erdr1 mRNA expression between B16F10 and B16F10 antisense IL-18 murine melanoma cell lines. (A) Total RNA was extracted from each cells. The RNA was reverse transcribed, and PCR was performed after reverse transcription with primers for Erdr1 or β-actin. PCR products were analyzed by 1.5% agarose gel electrophoresis. (B) Real time PCR analysis was used to detect Erdr1 mRNA expression in B16F10 and B16F10 antisense IL-18 cells. Data are expressed as the ratio of Erdr1 to β-actin mRNA expression. B16F10 antisense IL-18 cells highly expressed Erdr1 mRNA transcripts. A representative experiment of three performed is shown.

FIG. 2 shows Erdr1 overexpression induced by transfection of Erdr1 cDNA.

B16F10 cells were transfected with the Erdr1 cDNA by using lipofectamine as described in “Materials and Methods.” (A) RT-PCR analysis of Erdr1 mRNA expression. Total RNA was isolated from cells. Reverse transcription was performed and followed by PCR with oligonucleotides specific for Erdr1 or β-actin. PCR products were analyzed by 1.5% agarose gel electrophoresis. (B) Erdr1 western blot of B16F10 cells that transfected with indicated plasmids. Cell lysates containing equal amounts of protein were resolved by 12% PAGE and transferred onto Immuno-Blot PVDF membrane (Bio-Rad). The blot was incubated with anti-Erdr1 antibody or gamma tubulin antibody followed by incubation with peroxidase-conjugated secondary antibody. The antigen-antibody complexes were detected by an enhanced chemiluminescence system. Erdr1 plasmid transfected cells highly expressed of Erdr1 protein than empty vector transfected cells. A representative experiment of three performed is shown; Mock, negative control; Vector, vector transfectants; Erdr1, Erdr1 plasmid transfectants.

FIG. 3 shows Erdr1 overexpression inhibited cell migration and invasion.

(A) Cells that transfected with empty vector or Erdr1 plasmid were placed in the insert. Migration chamber was incubated for 12 h. Migrated cells were stained with 0.1% crystal violet solution. The stained cells were dissolved in 0.1% acetic acid. The OD value was measured at 570 nm. (B) Erdr1 or empty vector transfected cells were located onto matrigel coated well for 24 h. Invased cells were stained with crystal violet staining solution and staining level was measured at 570 nm. Erdr1 transfected group indicated inhibitory pattern of cell migration and invasion. These data are representative of three independent experiments. The data are reported as mean±SD. *P<0.01 vs control

FIG. 4 shows Expression of HSP90 inhibited by Erdr1 overexpression.

(A) RT-PCR analysis of HSP90 mRNA expression. Total RNA was isolated from cells. Reverse transcription was performed and followed by PCR with primer specific for HSP90 or β-actin. PCR products were analyzed by 1.5% agarose gel electrophoresis. (B) For quantitative analysis, Real time PCR was preformed. Data are expressed as the ratio of HSP90 to β-actin mRNA expressionA representative experiment of three performed is shown. The data are reported as mean±SD. *P<0.01 vs control. (C) HSP90 western blot of B16F10 cells by transfection with indicated plasmids as described in “Materials and Methods”. Briefly, cell lysates containing equal amounts of protein were resolved by 8% PAGE and transferred onto Immuno-Blot PVDF membrane (Bio-Rad). The blot was incubated with anti-HSP90 antibody or gamma tubulin antibody followed by incubation with peroxidase-conjugated secondary antibody. The antigen-antibody complexes were detected by an enhanced chemiluminescence system. Erdr1 transfected cells showed decreased HSP90 expression protein than empty vector transfected cells. A representative experiment of three performed is shown; Vector, vector transfectants; Erdr1, Erdr1 plasmid transfectants.

FIG. 5 shows Erdr1 inhibited cell motility is mediated ROI generation.

(A) Cells were transfected with the Erdr1 plasmid or empty vector for 24 h and then assayed for measuring ROI level by using DCFH-DA fluorescence by flow cytometry. Erdr1 transfected group showed lower ROI generation than vector control group. (□, vector transfectansts; ▪, Erdr1 transfectants) The data are reported as mean±SD. *P<0.01 vs control from three independent experiments. (B) Erdr1 transfected cells were treated with 0, 1, 5, 10 uM H2O2 for 24 h, and then HSP90 expression level was analyzed by western blot as described in “Materials and Methods”. The HSP90 expression level increased by adding hydrogen peroxide as dose dependent manners. A representative experiment of three performed is shown.

FIG. 6 shows Erdr1 over-expression suppressed melanoma metastasis in vivo.

(A) The B16F10 mouse melanoma tumors was established by I.V. injection of 5×104 vector or Erdr1 transfectants into C57BL/6 female mice. After two weeks, mice were sacrificed and the number of experimental visible lung metastasis was quantified. (B) Representative pictures of lungs from mice injected with B16F10 control cells or with cells transfected with Erdr1. The group injected with Erdr1 transfected cells significantly suppressed melanoma metastasis. A representative experiment of three performed is shown.

FIG. 7 shows that recombinant Erdr1 enhances the ability to kill cancer cells by mediating degranulation of natural killer cells. Human primary natural killer cells were subdivided into recombinant Erdr1-treated and non-treated groups. Then, after treatment with the degranulation inhibitor concanamycin A at 50 nM for 90 minutes or without treatment (No), K562 cells (human blood cancer cells) were incubated at 37° C. for 1 hour, and the ability of the natural killer cells to kill the blood cancer cells was investigated. The treatment with the recombinant Erdr1 resulted in enhanced ability of the natural killer cells to kill the blood cancer cells (K562), suggesting that degranulation is involved therein.

FIGS. 8 and 9 show that recombinant Erdr1 reduces viability of B-cell lymphoma cells. After treating Raji cells (human B-cell lymphoma cells) with recombinant Erdr1 at varying concentrations, cell viability was observed at 24, 48 and 72 hours (FIG. 8). Further, flow cytometry after treating with recombinant Erdr1 for 72 hours and then treating with annexin V/7AAD stain revealed that the treatment of the Raji cells with the recombinant Erdr1 result in increase of annexin V-positive cells (FIG. 9).

FIG. 10 shows that recombinant Erdr1 reduces mobility of cancer cells. Human gastric cancer cells (SNU-601) were treated with recombinant Erdr1 at concentrations of 0.1, 10 and 1000 ng/mL under usual culturing conditions and then incubated at 37° C. for 24 hours. Then, the cells were transferred to a transwell plate. After further incubating at 37° C. for 48 hours in a 5% CO₂ incubator to allow the cells to migrate, the cells that passed through the transwell plate and adhered to the bottom portion of the membrane were stained with crystal violet, the dye was dissolved with 10% acetic acid, and absorbance was measured using an absorbance detector (ELISA reader).

FIG. 11 shows a result of measuring expression of Erdr1 in normal tissue and melanoma tissue. Skin tissues of a healthy person and a melanoma patient were treated with anti-Erdr1 antibody and then compared by immunohistochemical staining. As seen from FIG. 11 (a), Erdr1 was expressed in the normal skin tissue (stained brown), whereas FIG. 11 (b) shows that Erdr1 was not expressed in the melanoma tissue.

For reference, techniques for the nucleotides and the proteins mentioned in the present invention are disclosed in the publication below (Maniatis et al., Molecular Cloning: A Laboratory Manual, Cold Spring Harbor Laboratory, Cold Spring Harbor, N.Y. (1982); Sambrook et al., Molecular Cloning: A Laboratory Manual, 2d Ed., Cold Spring Harbor Laboratory Press (1989); Deutscher, M., Guide to Protein Purification Methods Enzymology, vol. 182. Academic Press. Inc., San Diego, Calif. (1990)

### Advantageous Effects

As can be seen from the foregoing, Erdr1 of the present invention is negatively regulated by IL-18 expression and it suppresses migration, invasion and metastasis of cancer or tumor cell by expression of HSP90 and generation of ROI. And, an Erdr1 recombinant protein promotes NK-cell killing activity against cancer cell. Accordingly, Erdr1 of the present invention and an expression vector comprising polynucleotide encoding thereof and recombinant protein suppress cancer metastasis and bring an effect on activation of immune cells, and therefore can be useful for preventing and treating cancer.

### DESCRIPTION OF DRAWINGS

FIG. 1 shows that the level of Erdr1 is increased in B16F10 antisense IL-18 (B16F10/asIL18: B16F10 expressing antisense RNA for IL-18);

FIG. 2 shows that transfection with Erdr1 cDNA induces overexpression of Erdr1;

FIG. 3 shows that overexpression of Erdr1 suppresses cell migration and invasion;

FIG. 4 shows that expression of HSP90 is suppressed by overexpression of Erdr1;

FIG. 5 shows that the suppression of cell mobility by Erdr1 is mediated by ROI generation;

FIG. 6 shows that overexpression of Erdr1 suppresses melanoma metastasis in vivo (vector: empty vector-treated group, Erdr1: Erdr1 overexpressing vector-treated group);

FIG. 7 shows the effect of treatment with Erdr1 protein on cytotoxicity of natural killer cells;

FIG. 8 shows the effect of treatment with Erdr1 protein on viability of B-cell lymphoma cells;

FIG. 9 shows a result of investigating whether treatment with Erdr1 protein induces apoptosis of B-cell lymphoma cells;

FIG. 10 shows the effect of Erdr1 protein on mobility of gastric cancer cells; and

FIG. 11 shows a result of investigating whether Erdr1 protein is expressed in normal and melanoma skin tissue.

### MODE FOR INVENTION

Hereafter, the present invention will be described in detail by the examples. It is to be understood, however, that these examples are for illustrative purpose only and are not constructed to limit the scope of the present invention.

<Materials and Methods>

1. Cell Culture

The murine melanoma cell lines, B16F10 were cultured in DMEM that was supplemented with 2 mM 1-glutamine, 100 units/ml penicillin, 100 μg/ml streptomycin and 10% heat-inactivated fetal bovine serum. The cells were cultured at 37° C. in a humidified atmosphere that contained 5% CO₂ in air. These cell lines were used for experiments while they were in the log phase of growth.

2. Construction of Erdr1 cDNA

For construction of the mouse Erdr1 expression vector, the complete coding sequences of Erdr1 were isolated by polymerase chain reaction amplification from B16F10 cell cDNA using primers based on the known sequences (Genbank Accession No: NM_{—}133362). The Erdr1 cDNA fragments were digested with EcoR I and Xho I, and ligated into pcDNA3.1(+) (Invitrogen). Plasmid DNA used for transfection was prepared by endo-free plasmid Maxi kit (Qiagen). For each plasmid the A260/A280 was determined spectrophotometrically and was typically between 1.8 and 2.0. Absence of RNA and genomic DNA was checked by gel electrophoresis.

3. Transiently Transfection of Erdr1 cDNA

Cells were cultured in antibiotics free medium and reached 90% to 95% confluence, cells were transfected with plasmid containing the full length murine Erdr1 cDNA or empty vector, pcDNA3.1(+) using Lipofectamine™ 2000 (Invitrogen, Carlsbad, Calif.) according to the manufacture's recommendation. Briefly, each plasmid, empty vector and Lipofectamine™ 2000 were diluted in serum-free Opti-MEM medium, left at room temperature for 5 minutes, mixed gentely, and incubated for 20 minutes at room temperature. The mixture was then added to cultured cells, and incubated at 37° C. in a humidified atmosphere that contained 5% CO₂ incubator. After for 24 hours of incubation, transfection effect was confirmed by RT-PCR and western blot prior to function study.

4. RT-PCR

Total RNA was extracted from B16F10 melanoma cells using Trizol, according to the instructions of the manufacturer. After reverse transcription, the cDNA was incubated with following primers: (1) Erdr1, sense 5′-CAGTGATGTCACCCACGAAA-3′ (SEQ ID NO:3), antisense 5′ GGCATTT CTGTACGCAGTCA-3′ (SEQ ID NO:4), (2) HSP90, sense 5′-TCACCCACACTGTGCCCATCTACG-3′ (SEQ ID NO:5), antisense 5′-CAGCGGAACCGCTCATTGCCAATG-3′ (SEQ ID NO:6), (3) β-actin, sense 5′-TCACCCACACTGTGCCCA TCTACG-3′ (SEQ ID NO:7), antisense 5′-CAGCGGAACCGCTCATTGCCAATG-3′ (SEQ ID NO:8), for PCR amplification. The cycling conditions were over 25 cycles denaturing. (94° C., 30 sec), annealing (55° C., 30 sec), and extension (72° C., 30 sec), with a final extension at 72° C. for 10 min.

5. Western Blot Analaysis

The polyclonal anti-Erd1 antibody were generated in rabbits by using a. C-terminal Erdr-1 peptide of 36-51 amino acids (C-RAPRPPRHTRHTRHTR-NH2, SEQ ID NO:9) for immunization according to standard protocols. Cells were washed twice with ice-cold PBS and extracted in ice-cold lysis buffer [50 mM Tris-HCl (pH 7.4), 1% NP-40, 0.25% Deoxycholic acid sodium salt, 150 mM NaCl, 1 mM EDTA, and a protein inhibitor cocktail. After collecting the cell lysate, protein quantity was determined using a Bradford assay (Bio-Rad, Hercules, Calif.). An equal volume of protein was separated by 12% SDS-PAGE under reducing conditions and transferred to a PVDF membrane (Bio-Rad, Hercules, Calif.). The membrane was blocked with 5% non-fat dried milk for 1 h, and then incubated with Erdr1 antibodies, HSP90 antibodies (Santa-cruz) or anti-tubulin antibodies (Cell Signaling Technology) for overnight. After washing, the membrane was incubated for 1 h with either goat anti-rabbit IgG antibodies or donkey anti-goat IgG antibodies conjugated with biotin. The membrane was incubated for 30 min with horseradish peroxidase (Amersham Pharmacia Biotech, Buckinghamshire, UK). Each of the proteins was detected using an Amersham ECL system (Amersham Pharmacia Biotech).

6. Invasion and Migration Assay

Migration assay was performed using a Transwell chamber (Costar, Cambridge, Mass., USA) with 8 μm pore polycarbonate filters. Briefly, the cells were suspended into upper chambers 100 μl of serum-free media at a final concentration of 5×10⁵ cells/ml. Medium containing 10% FBS was placed into the lower chamber. After incubation for 12 h, the cells that migrated through the pores in the membrane were Stained with a staining solution (0.1% crystal violet in ethanol). The stained cells were dissolved in 10% acetic acid. The O.D. values at 570 nm were measured using an ELISA reader (Molecular Devices, Sunnyvale, Calif., USA). Invasion assay was determined using Matrigel invasion chambers (BD) which pre-coated with matrigel matrix. The cells were introduced into upper chambers 100 μl of serum-free media at a final concentration of 1×10⁶ cells/ml. Lower compartment contained 10% FBS medium. After invasion for 24 h, Analysis of invasion ability performed same as is migration assay.

7. Measurement of Intracellular ROI Levels

ROI production was determined using the fluorescent dye 2′,7′-dichlorofluorescein dicetatate (DCFH-DA; sigma) whose fluorescence intensity is correlated with cellular oxidative stress. After cells were treated with 50 μM DCFH-DA for different time periods at 37° C. and 5% CO₂, then washed twice or three times with PBS and resuspended in PBS at 4° C. Intracellular ROI generation was analyzed by fluorescence intensity (FL-1, 530 nm) FACS Calibur (Becton Dickinson, Sunnyvale, Calif.) using CellQuest software.

8. In Vivo Tumorigenecity Model

C57BL/6 mice aged 5 weeks were purchased from Cental Lab. Animal Inc, Korea. It maintained for 1 week before starting of the experiments. To examine the metastatic potential, we performed intravenous (i.v.) inoculation. B16F10 cells (5×10⁴ cells/100 ul PBS) transfected with or without Erdr1 were injected in the lateral tail vein (n=10). Two weeks later, mice were sacrificed and the lungs were excised for counting the number of colonies. Experiments were performed as two or three independent.

9. Effect of Erdr1 on Killing Ability and Degranulation of Natural Killer (NK) Cells

Human natural killer cells were obtained from peripheral blood mononuclear cells (PBMCs) isolated from the peripheral blood of a healthy volunteer using a natural killer cell isolation kit (MACS, USA). This method involves specific binding of T cells, B cells, stem cells, dendritic cells, monocytes, granulocytes, red blood cells, etc. onto magnetic microbeads using an antigen-binding agent, followed by removal using magnetic field. Thus isolated pure NK cells were transferred to a PRMI1640 medium (Gibco, USA) containing 2 mM 1-glutamine and 10% fetal bovine serum (Gibco, USA) at 1.5×10⁶/mL and then treated with 10 ng/mL recombinant Erdr1 for 72 hours. Before measuring the killing ability of the NK cells by immunofluorescence staining (FACS staining), the cells had been pretreated with the degranulation inhibitor concanamycin A (Sigma, USA) at 50 nM for 90 minutes, and K-562 (human leukemia cell line) cells stained with carboxyfluorescein diacetate succinimidyl ester (CFSE, Invitrogen, USA) had been cultured at 37° C. for 1 hour as target cells. After treating with the 7AAD stain (BD Bioscience, USA) for 5 minutes in order to specifically stain only the dead cells, the killing ability of the NK cells was investigated by flow cytometry.

10. Expression of Erdr1 in Melanoma Tissue

The difference in Erdr1 expression in normal and melanoma tissues was investigated by an immunohistochemical method. The experiment was approved by the Ethics Committee of the Catholic University and carried out in accordance with the Helsinki Declaration. Skin tissues acquired from 5 healthy people and 30 melanoma patients were fixed with formalin and prepared into paraffin sections. The tissue sections were treated with anti-Erdr1 polyclonal antibody (1:1000) at 4° C. for 12 hours and then subjected to the streptavidin-biotin-peroxidase detection using a Cap-plus detection kit (Invitrogen, Camarillo, Calif., USA).

<Result>

1. Erdr1 Increased by Reduction of IL-18 on B16F10 Mouse Melanoma Cells.

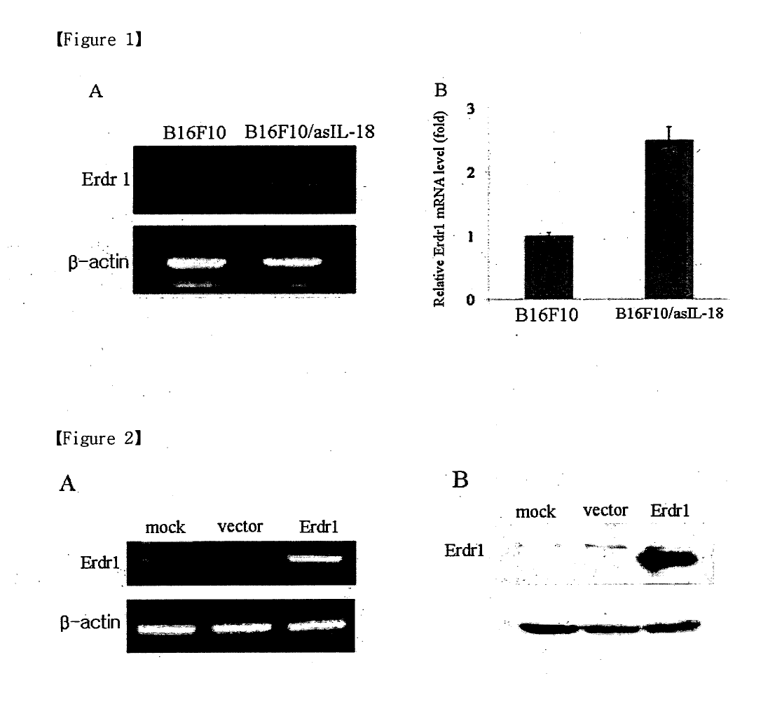

As a first step of present study, we examined whether Erdr1 is regulated by IL-18 in melanoma cell lines, B16F10 by using B16F10/IL-18 antisense transfectants. In previously our studies, we established B16F10/IL-18 antisense transfectants, which express a lower level of IL-18 by transfection with IL-18 antisense cDNA. The expression level of Erdr1 protein and its mRNA transcripts was determined using western blot analysis and RT-PCR, respectively. FIG. 1 shows that the level of Erdr1 was significantly increased on B16F10/IL-18 antisense transfectants compare to B16F10 wild type. This suggests that Erdr1 is negatively regulated by IL-18 expression. Because of IL-18 showed pro-cancer effects in various reports, we next asked whether Erdr1 could act as anti-cancer factor.

2. Over-Expression of Erdr1 Reduced Cell Migration and Invasive Ability

To evaluate the effect of Erdr1 in murine melanoma cell line, B16F10, we preformed transiently transfection the murine Erdr1 cDNA in B16F10 cells then these cells were used for subsequent studies. RT-PCR and Western blot analysis with a specific antibody against Erdr1 revealed that Erdr1 transfected cells highly expressed the Erdr1. As expected, Erdr1 could not be detected in cells transfected with an empty vector (FIG. 2). Thus, we used vector transfected cells as negative controls in this study.

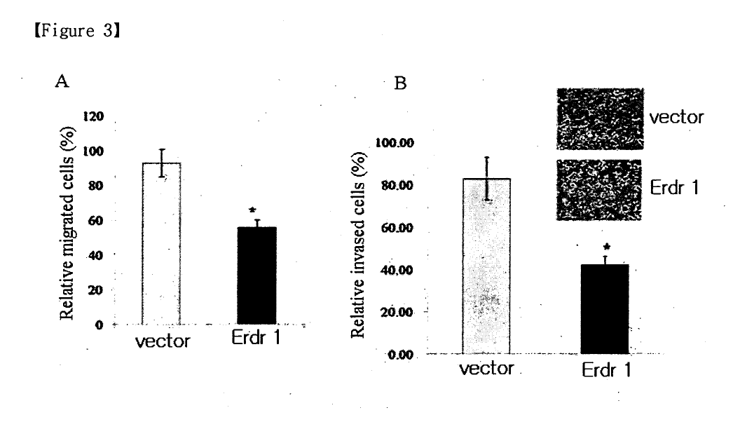

Cell motility is key step in tumor metastatic process and an initial step in cell invasion is migration ability. In order to determine if the Erdr1 regulate migration ability of melanoma cells, transwell migration assay was then performed. FIG. 3A shows that the migration ability of the Erdr1 over-expression group was lower about 50% than that of the vector transfection group. Next, to determine the possible role of Erdr1 in the invasiveness of melanoma cells, we used a matrigel coated transwell invasion assay. As shown FIG. 3B, invasion ability is significantly suppressed about 60% by Erdr1 overexpression. These data suggested that Erdr1 inhibits the migration and invasive capacity of B16F10 cells, and Erdr1 act potent suppressor melanoma motility.

3. Melanoma Cells Over-Expressing Erdr1 Inhibited Heat Shock Protein 90 Expression and ROI Signaling is Closely Realted.

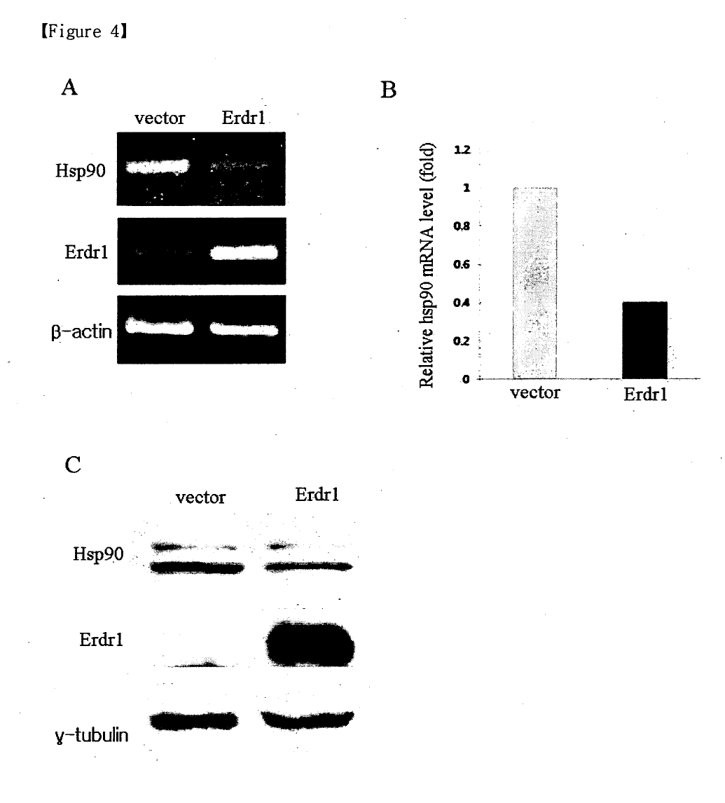

Next, we tried to find the related factor by which Erdr1 is able to regulate cell motility. Heat shock protein (HSP), reported as pro-cancer factor and induced active invasion ability in cancer cells. Especially, HSP90 is reported that its expression is enhanced in advance malignancy melanoma and its inhibitor acts as anticancer effects. In FIG. 4, HSP90 decreased pattern was shown on Erdr1 overexpressed group in mRNA and protein levels. On the basis of these result, we can conclude that Erdr1 inhibit expression of HSP90, and this might be a candidate mechanism Erdr1 suppresses motility of melanoma cells.

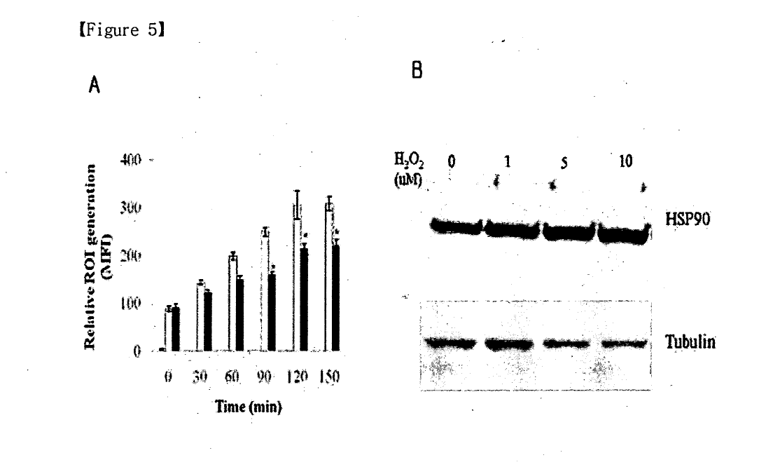

It is well known that ROI acts as oxidative stress, play an important role in the intracellular signal transduction pathway in various cancer cell. Our data has previously shown that ROI is mediated on IL-18 enhanced migration ability in melanoma cells. To investigate whether ROI is involved in the pathway of Erdr1-reduced motility, the ROI levels were measured by performing FACS analysis. Cells that no treated with DCFHDA were used for negative control to check for authentic intracellular ROI generation. FIG. 5 show that ROI levels were markedly increased by Erdr1 transfection in a time dependent manner. To investigate that reduced ROI generation is also related with down-regualtaion of HSP90, B16F10/Erdr1 transfectant cells were treated for 24 h with hygrogen peroxide (H2O2), which is one of the major ROI. And then HSP90 expression level was measured. The reduced HSP90 expression level by Erdr1 transfection was recoved as treatment of hygrogen peroxides. It indicated that inhibited melanoma migration by Erdr1 occurs via generation of ROI. And reduced ROI generation affect to HSP90 down-regulation.

4. Erdr1 Over-Expression Suppressed Tumor Metastasis and Induced Prolonged Survival

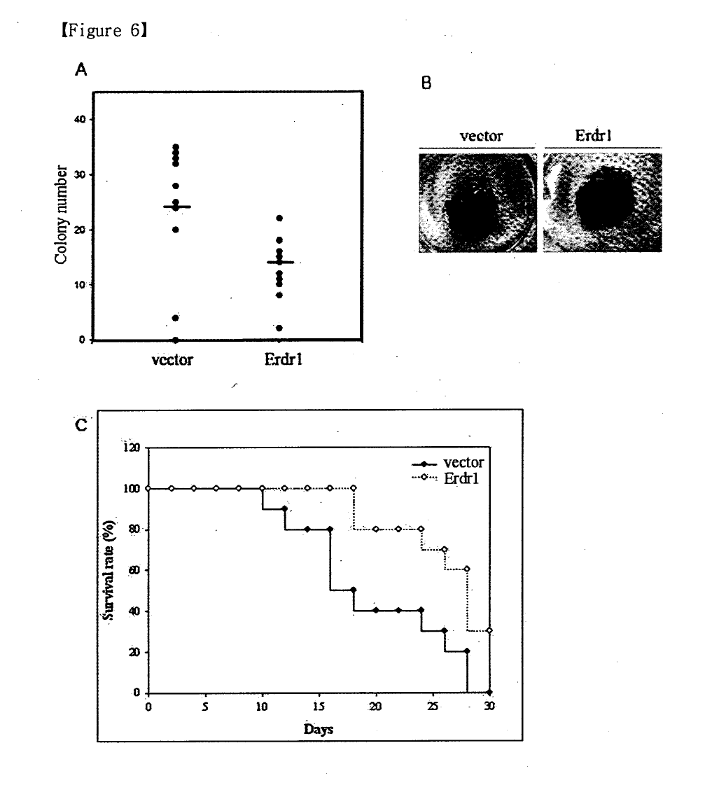

To examine whether Erdr1 affect on melanoma metastasis in vivo, C56/BL6, syngenic mice used. Melanoma cells transfected with Erdr1 cDNA or with an empty vector were intravenously injected into the tail vein of mice and the number of lung metastasis was counted. As shown in FIG. 6A. lung colonization was significantly inhibited in Erdr1 transfected group. In addition, the survival rate was prolonged in mice implanted with Erdr1 transfected group compared with those injected with vector control group (FIG. 6B). Twenty days after injection of melanoma cells, the survival rate of Erdr1 overexpression group is 2 fold higher than that of vector control group. These in vivo data indicate that Erdr1 effectively suppressed metastatic ability of melanoma, accordingly it suggested as novel anti-cancer factor.

5. Treatment with Recombinant Erdr1 Enhances NK Cells' Ability to Cancer Cells by Mediating Degranulation.

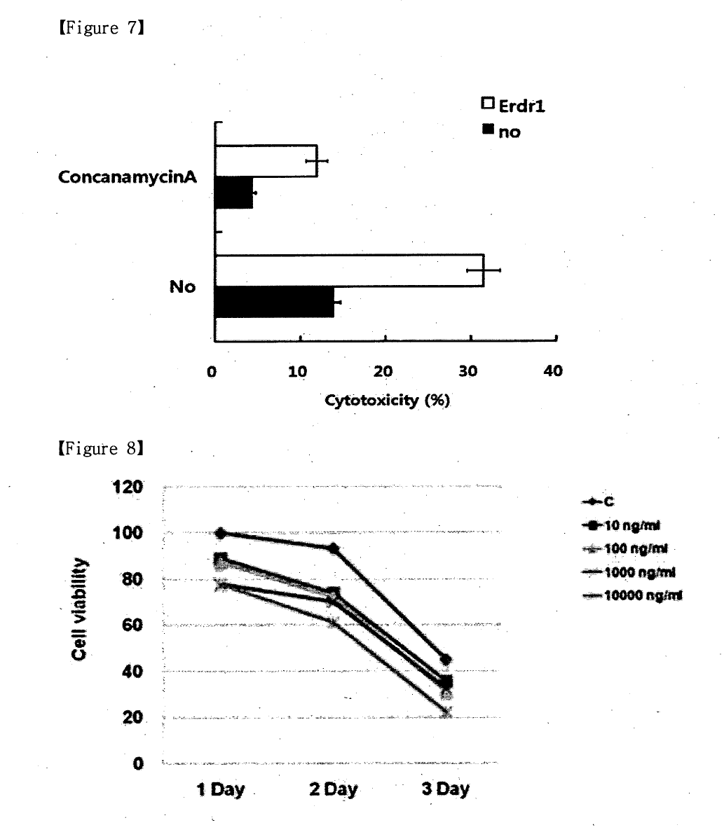

In order to study the anticancer effect of recombinant Erdr1, experiment was carried out to investigate NK cells' killing ability and its mechanism. Before measuring the killing ability of the NK cells by immunofluorescence staining (FACS staining), the cells had been pretreated with the degranulation inhibitor concanamycin A at 50 nM for 90 minutes, and K562 (human leukemia cell line) cells had been cultured at 37° C. for 1 hour as target cells. After treating with the 7AAD stain in order to specifically stain only the dead cells, the killing ability of the NK cells was investigated by flow cytometry. As seen from FIG. 7, the treatment with the recombinant Erdr1 resulted in enhanced ability of the NK cells to kill the leukemia cells (K562). It was found out that degranulation is involved therein.

6. Treatment with Recombinant Erdr1 Induces Death (Apoptosis) of Human B-Cell Lymphoma Cells.

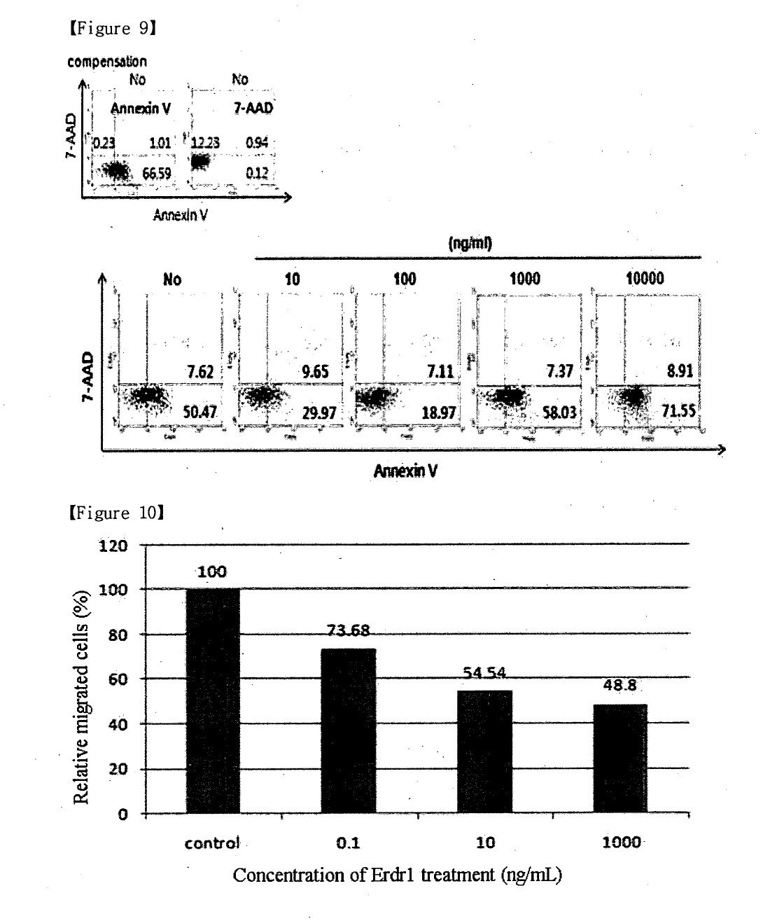

In order to investigate the anticancer effect of recombinant Erdr1 against lymphoma, apoptosis of B-cell lymphoma cells was studied. Raji cells (human B-cell lymphoma cells) were treated with recombinant Erdr1 at various concentrations, and cell viability was examined at 24, 48 and 72 hours by staining with trypan blue and then counting living and dead cells. As seen from FIG. 8, the Raji cells treated with the recombinant Erdr1 showed temperature-dependent decrease of viability at each time. In order to confirm whether the decrease in viability is due to apoptosis, flow cytometry was performed after treating with the recombinant Erdr1 for 72 hours and then treating with the annexin V/7AAD stain. When apoptosis occurs, the phospholipid phosphatidylserine normally found on the cytosolic surface of the cell membrane moves to the extracellular surface. Since annexin V binds avidly to the phosphatidylserine, annexin V is observed positive, but 7AAD is observed negative in the early stage of apoptosis. Through the experiment, it was observed that the treatment of the Raji cells with the recombinant Erdr1 results in increased annexin V-positive cells (FIG. 9), suggesting that the recombinant Erdr1 induces apoptosis of the B-cell lymphoma cells.

7. Treatment with Recombinant Erdr1 Reduces Mobility of Cancer Cells.

In order to investigate the effect of the recombinant Erdr1 on mobility of cancer cells, experiment was carried out using human gastric cancer cells SNU-601 (Korean Cell Line Bank, KCLB No. 00601). First, after treating the cells with the recombinant Erdr1 at three concentrations of 0.1, 10 and 1000 ng/mL under usual culture conditions, the cells were cultured for 24 hours at 37° C. Then, the cells were transferred to a transwell plate consisting of a membrane through which the cells can pass, and the mobility of the cells was measured after incubation for 48 hours at 37° C. in a 5% CO₂ incubator. The cells that passed through the transwell plate and adhered to the bottom portion of the membrane were stained with crystal violet, the dye was dissolved with 10% acetic acid, and absorbance was measured using an absorbance detector (ELISA reader). As a result, the gastric cancer cells of the group treated with the recombinant Erdr1 showed decreased mobility (FIG. 10).

8. Erdr1 is not Expressed in Melanoma Tissue.

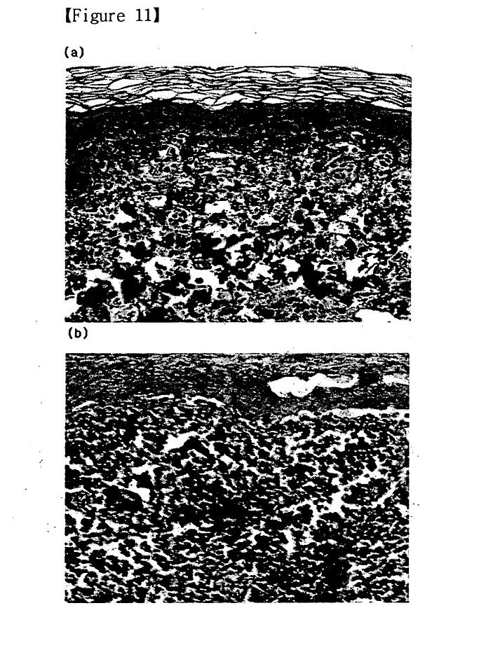

It was investigated whether Erdr1 is expressed in skin tissues of a healthy person and a melanoma patient by treating them with anti-Erdr1 polyclonal antibody (1:1000) as primary antibody at 4° C. for 12 hours and then detecting by the streptavidin-biotin-peroxidase detection method using a Cap-plus detection kit (Invitrogen, Camarillo, Calif., USA). As seen from FIG. 11 (a), Erdr1 was expressed in the normal skin tissue (stained brown), whereas FIG. 11 (b) shows that Erdr1 was not expressed in the melanoma tissue.

### INDUSTRIAL APPLICABILITY

As can be seen from the foregoing, Erdr1 of the present invention is negatively regulated by IL-18 expression and it suppresses migration, invasion and metastasis of cancer or tumor cell by expression of HSP90 and generation of ROI. And, an Erdr1 recombinant protein promotes NK-cell killing activity against cancer cell. Accordingly, Erdr1 of the present invention and an expression vector comprising polynucleotide encoding thereof and recombinant protein suppress cancer metastasis and bring an effect on activation of immune cells, and therefore can be useful for preventing and treating cancer.

1. A method for preventing or treating cancer, comprising administering an effective amount of an Erdr1 (erythroid differentiation regulator 1) polypeptide or an expression vector comprising polynucleotide encoding the polypeptide to a subject in need thereof.

2. The method according to claim 1, wherein the Erdr1 (erythroid differentiation regulator 1) polypeptide comprises an amino acid sequence represented by SEQ ID NO: 1.

3. (canceled)

4. The method according to claim 1, wherein the polynucleotide comprises a nucleotide sequence represented by SEQ ID NO: 2.

5. The method according to claim 1, wherein the cancer is selected from the group consisting of leukemia, malignant melanoma, colon cancer, lung cancer, liver cancer, stomach cancer, esophagus cancer, pancreatic cancer, gall bladder cancer, kidney cancer, bladder cancer, prostate cancer, testis cancer, cervical cancer, endometrial carcinoma, choriocarcinoma, ovarian cancer, breast cancer, thyroid cancer, brain tumor, head or neck cancer, skin cancer, lymphoma and aplastic anemia.

6-17. (canceled)

18. A method for screening agents for regulating cancer metastasis or cancer cell migration comprising:
(a) contacting an Erdr1 (erythroid differentiation regulator 1) polypeptide to a test agent in the presence of the test agent;
(b) selecting the test agent which changes Erdr1 activity by measuring Erdr1 activity; and
(c) testing whether the selected test agent regulates cancer metastasis or cancer cell migration.

19. The method according to claim 18, wherein the cancer is selected from the group consisting of leukemia, malignant melanoma, colon cancer, lung cancer, liver cancer, stomach cancer, esophagus cancer, pancreatic cancer, gall bladder cancer, kidney cancer, bladder cancer, prostate cancer, testis cancer, cervical cancer, endometrial carcinoma, choriocarcinoma, ovarian cancer, breast cancer, thyroid cancer, brain tumor, head or neck cancer, skin cancer, lymphoma and aplastic anemia.

20. The method according to claim 1, wherein preventing or treating cancer is mediated by apoptotic cell death or inhibition of cancer cell metastasis.

21. A method for stimulating NK (natural killer) cell cytotoxicity, comprising contacting said NK cells with an Erdr1 (erythroid differentiation regulator 1) polypeptide or an expression vector comprising polynucleotide encoding the polypeptide.

22. The method according to claim 21, wherein the Erdr1 (erythroid differentiation regulator 1) polypeptide comprises an amino acid sequence represented by SEQ ID NO: 1.

23. The method according to claim 21, wherein the polynucleotide comprises a nucleotide sequence represented by SEQ ID NO: 2.

24. A composition for diagnosing cancer, comprising an antibody specific for an Erdr1 (erythroid differentiation regulator 1) polypeptide as an active ingredient.

25. The composition according to claim 24, wherein the cancer is selected from the group consisting of leukemia, malignant melanoma, colon cancer, lung cancer, liver cancer, stomach cancer, esophagus cancer, pancreatic cancer, gall bladder cancer, kidney cancer, bladder cancer, prostate cancer, testis cancer, cervical cancer, endometrial carcinoma, choriocarcinoma, ovarian cancer, breast cancer, thyroid cancer, brain tumor, head or neck cancer, skin cancer, lymphoma and aplastic anemia.

26. A method for diagnosing cancer in a subject suspected of having cancer, comprising:
(a) contacting the composition of claim 24 with a biological sample from the subject suspected of having cancer;
(b) measuring and comparing a level of an antigen-antibody complex resulted from the binding between the antibody and the Erdr1 (erythroid differentiation regulator 1) polypeptide in the contacted biological sample and a control sample; and
(c) determining the presence of cancer when the level of antigen-antibody complex in the biological sample is decreased as compared to the level in the control sample.


SEQUENCE LISTING (9 sequences)

SEQ ID NO: 1 - protein, 177 aa, Mus musculus
MPTGRTDGRTHGRTPQGRKPAPTAPLHPPQHTGHTRAPRPPRHTRHTRHTRQAGQAHASAGPAAPATQTRTSRRGQDVHPPRSRCMCHRPSPRWTDGRTHARRQRPPVTAAAHSDVTHESTHVEADAVVKMSLPSPQDGRTDSTRCACRRGRQDGAILTEEGARQQGLTAYRNAPPQ

SEQ ID NO: 2 - DNA, 801 bp, Mus musculus
gtccgttctcttttagccgcagctatggtttctgccctaattattcttgtccttatttgtaatttaattcttaatttaatttaatttataattttgttgtaagtttctctgtgggcgtgaatggaaagtctaacccgtgtttctctgttcagcgtccgccggtcacggccgccgcccccagcgacgtcacccacacgcgcagaagcggacgccgcggtcaagatgtctctgccatgcccacgggacgcacggacggacggacgcacggacggactccacaaggtaggaagcctgcgccgaccgcaccgctgcacccaccacagcacacaggacacacgcgggccccgcgcccgcccaggcacacgcggcacacacggcacacacggcaggcaggccaggcacacgcatccgcaggacccgccgcacccgccacgcagacacggacgagccgccgcggtcaagatgttcacccgccgcggtcaagatgtatgtgccaccgaccctcgccccgctggacggacggacggacgcacgcacgccgtcagcgtccaccggtcactgccgccgcccacagtgatgtcacccacgaaagcacacacgtagaagcggacgccgtggtcaagatgtctctgccatccccacaggacggacggacggactccacaaggtgcgcgtgtcgccgaggccgccaggacggagcgattctcacggaggaaggagcacgccaacagggcctgactgcgtacagaaatgccccccctcaataaaattgcagttgaaatggaaaaaaaaaaaaaaaaa

SEQ ID NO: 3 - DNA, 20 bp, Artificial Sequence
  Erdr1 sense
cagtgatgtcacccacgaaa

SEQ ID NO: 4 - DNA, 20 bp, Artificial Sequence
  Erdr1 antisense
ggcatttctgtacgcagtca

SEQ ID NO: 5 - DNA, 20 bp, Artificial Sequence
  HSP90 sense
tcggcaccatagccaaatct

SEQ ID NO: 6 - DNA, 20 bp, Artificial Sequence
  HSP90 antisense
aaccgacaccaaactggcca

SEQ ID NO: 7 - DNA, 24 bp, Artificial Sequence
  beta-actin sense
tcacccacactgtgcccatctacg

SEQ ID NO: 8 - DNA, 24 bp, Artificial Sequence
  beta-actin antisense
cagcggaaccgctcattgccaatg

SEQ ID NO: 9 - protein, 16 aa, Artificial Sequence
  Erdr1 c-terminal peptide
RAPRPPRHTRHTRHTR